(19) United States
(12) Patent Application Publication
(10) Pub. No.: US 2007/0275013 A1
(43) Pub. Date: Nov. 29, 2007

(54) Vaccine and Method of Use

(76) Inventor: Victor Anomah Ngu, Yaounde (CM)

Correspondence Address: Thomas Moga;Butzel Long, STONERIDGE WEST, 41000 WOODWARD AVENUE, BLOOMFIELD HILLS, MI, 48304, US

(21) Appl. No.: 10/573,724

(22) PCT Filed: Apr. 8, 2005
(86) PCT No.: PCT/IB05/02424

§ 371 Date: Mar. 26, 2007

Related U.S. Application Data
(60) Provisional application No. 60/561,408, filed on Apr. 12, 2004.

(51) Int. Cl. A61K 39/12; A61P 31/04; A61P 31/12; A61K 39/21

(52) U.S. Cl. 424/208.1; 424/184.1; 424/204.1; 424/207.1; 424/227.1; 424/228.1; 424/230.1; 424/231.1; 424/234.1; 424/248.1; 424/269.1; 424/274.1

(57) ABSTRACT

A therapeutic vaccine is prepared by a method which includes the steps of extracting, with a lipid-extracting solvent, a biological fluid obtained from a person or animal infected with a lipid-containing infectious organism, the biological fluid containing the lipid-containing infectious organism, and the extraction producing an aqueous phase and a lipid-containing phase, said aqueous phase containing the infectious organism with the lipid substantially removed, and separating the aqueous phase from the lipid-containing phase. A leukocyte fraction is isolated from the blood of the person or animal, the isolation being conducted so that the leukocyte fraction is substantially without plasma, free lipid-containing infectious organism and free antibodies to the lipid-containing infectious organism, and at least some of the aqueous phase is combined with at least some of the leukocyte fraction to produce the vaccine.

### RELATED APPLICATION

This application claims the benefit of U.S. Provisional Patent Application No. 60/561,408 filed on Apr. 12, 2004, the entire disclosure of which is incorporated by reference.

### FIELD OF THE INVENTION

The present invention relates to a therapeutic vaccine and to a prophylactic vaccine, to a method of making a therapeutic vaccine and to a method of making a prophylactic vaccine, to a method of treating a person or animal infected with a lipid-containing infectious organism, to a substance or composition for use in a method of treatment of an infection caused by a lipid-containing infectious organism, to a substance or composition for use in vaccinating a person or animal against an infection caused by a lipid-containing infectious organism, to the use of a substance or composition in the manufacture of a medicament for use in the treatment of an infection caused by a lipid-containing infectious organism and to the use of a substance or composition in the manufacture of a medicament for use as a vaccine for the therapy or prophylaxis of an infection caused by a lipid-containing infectious organism.

### BACKGROUND OF THE INVENTION

Tropical Africa has been regarded as the home of HIV/AIDS, malaria, filariasis, tuberculosis and a myriad of other chronic tropical and parasitic infections. The question which arises is why are these infections chronic and why can the immune systems of infected persons not deal effectively with them.

Infectious diseases have caused enormous and prolonged human suffering and death throughout man's history. Certain infectious agents cause chronic infections from the start. The diseases they cause in man include for example, tuberculosis, leprosy, mycoses at various sites in the body, malaria, Onchocerca volvulosis and microfilaria that cause “river blindness.” The viruses include the well-known human immune deficiency virus (HIV), the human T-lymphocyte leukemia virus (HLTV1), the hepatitis B virus, the hepatitis C virus, the herpes simplex virus, the cytomegalo virus (CMV), the Epstein-Barr virus (EBV) of Burkitt's Lymphoma and other viruses that cause lymphomas and other malignant tumor disease.

Various animal species have similar or analogous microorganisms and viruses that cause chronic infections and tumors in them, for example bovine tuberculosis, retroviruses such as SIV in monkeys, ‘scrapies’ in sheep, or malignant tumors in chicken, mice, cats and cattle. This list is not exhaustive for humans or animals.

Hereafter the term “microorganism” should be understood to include bacteria, fungi, and parasites which affect humans and animals but to exclude viruses.

Without being bound by theory, the following is proposed as the basis for, and the understanding of, the invention. Most microorganisms that cause chronic infections in man must invade the human body from the outside. Before invading the body, they would all have had a separate and independent existence and evolution that has lasted perhaps millions of years in the earth's environment. In the process of transmission from one person to another, these microorganisms would again frequently have had to survive as independent agents for at least a short period in the environment before gaining access to a new host. They have all therefore been exposed to and have survived in the environment at some point in their evolution.

The earth's environment, to which these microorganisms have been exposed, is hostile in some ways to all forms of life because the sun, a constant part of our environment, dehydrates all living things exposed to it. The sun is particularly hostile to microorganisms because the very small amount of body water in such organisms can easily be lost by dehydration and this can cause the death of the organism. Microorganisms that have managed to survive in the earth's environment under the sun are therefore mutants that have acquired the ability to retain and conserve the water within them.

Lipids and lipid-like substance are relatively resistant to dehydration and most living surfaces exposed to the sun's rays or heat have a lipid material on them to reduce water losses. Microorganisms that have survived as independent agents in the earth's environment are therefore mutants that have acquired a lipid covering to stop or reduce water loss from their surfaces. The amount of the lipid covering on the microorganism will clearly determine its capacity to survive in the environment. Microorganisms that have a thick lipid covering can survive better than those with a thin lipid covering and the varying capacities of most microorganism to survive in the earth's environment as independent agents are related to the varying thicknesses of their lipid coverings. The tubercle bacillus, for example, can survive for about a year in domestic dust and is known to have a very thick lipid coat. This characteristic is clearly demonstrated in the Zeihl Nilsson stain.

Protective lipid coats that prevent water loss from the surface of microorganisms cover over, and in some way eventually hide, the true antigens which confer identity, specificity, or uniqueness to the microorganism concerned. When true and recognizable antigens from any source gain access to the body, as a first line of defense, natural killer cells normally produce cytokines that immediately destroy them. As a second line of defense, the long-term immune responses of the body (by antibodies and cell-mediated immune responses) will determine whether or not these true antigens persist in the body.

When a microorganism with a lipid covering invades the body in a normal infection, because of the inaccessibility of the true antigens beneath the lipid coats, the immediate response by cytokines and the long-term and effective immune responses to the microorganisms do not occur or occur very poorly. In contrast, the immune responses provoked by the lipid coat on the surface of the microorganism are not related to the true antigens beneath and will not seriously affect them. The microorganisms therefore survive in the body as chronic infections. By masking their true antigens with a lipid coat, microorganisms have therefore been able to conserve their body water and ensure their survival first in the environment and later to escape from the immune response of the body and so persist as chronic or as repeated infections.

The lipid coat of microorganisms can be stripped off in vitro with lipid solvents such as chloroform or ether to expose their true antigens. The duration of action of the lipid solvent will depend on the amount and nature of the lipid coat to be removed. Once exposed, the true antigens of the microorganisms can be re-injected as a kind of vaccine into an uninfected body. They will elicit from the body the immediate response of natural killer cells with cytokines and later by long-term immune responses with antibodies. When confronted by the same microorganism in a subsequent infection, these cytokines and immune responses will penetrate the lipid coat and destroy the true antigens beneath. In this way the vaccinated body is protected from the infecting microorganisms.

Experience has shown that for a preventive vaccine to achieve full immune protection of an uninfected body, it is necessary that:
the vaccine has exposed true antigens that are related to those of the microorganism concerned and that these true antigens are accessible to the immune system;
the person to be vaccinated has a competent immune system; and
there is no pre-existing infection or antibodies to the microorganism concerned in the serum of the body to be vaccinated.

An established infection may persist or be repeated because the true antigens of the microorganism concerned have never been exposed to the immune system and there are not antibodies to the true antigens in the person. In such a case, the exposed true antigens on the vaccine will provoke the immediate production of cytokines and the substances that penetrate the infectious microorganism and kill it beneath its lipid covering. The amount of cytokines produced and the numbers of microorganisms killed will depend on the quantity of the vaccine used and the numbers of available natural killer cells. In addition, the true antigens of the vaccine will provoke for the first time immune responses that will reinforce and eliminate the microorganism. Such a vaccine injected directly into the infected person in sufficient amounts and at suitable intervals will serve as a therapeutic vaccine.

In all chronic infections, however, demonstrable antibodies to the microorganism are normally present in the infected body. The continued infection in spite of these antibodies is proof of their irrelevance or incompetence. Such antibodies, moreover, may immobilize any vaccine related to the microorganism that is introduced directly into the infected body. In that case, one must carry out a form of immunotherapy and provoke new and effective immune responses that would completely destroy and prevent further infections by the same microorganisms.

### BRIEF SUMMARY OF THE INVENTION

The present invention provides a new vaccine which is prepared from the microorganisms of a current infection by exposing their true antigens by removing their lipid coating with lipid solvents. With a competent immune system, it is possible to artificially reproduce in vitro for the infected person conditions that are similar to those for a preventive vaccination in a normal uninfected person.

According to the method of the invention, peripheral leukocytes with competent immune cells taken from the infected person are washed free of all antibodies. The washed leukocytes contain competent immune cells not committed to the current infection and free to react to new antigens. The washed cells are suspended in vitro in a medium that contains the infected person's serum from which the immunoglobulin or antibodies have been removed. The situation in vitro is now similar or analogous to that seen in an uninfected person before an infection (cord serum without antibodies to the microorganism can replace the person's serum). The vaccine is added to the medium and incubated with the immune competent cells and then the mixture is re-injected into the infected person. The re-injected cells and their descendants would continue in vivo what was started in vitro and the new immune response provoked would progressively and completely kill the microorganisms. This would, paradoxically, achieve a kind of preventative vaccination of the infected person similar to injecting the vaccine directly into an uninfected person. The use of the vaccine in an established infection in this manner is immunotherapy of the infection. A normal preventive vaccine is also a kind of immunotherapy that is administered to an uninfected person in advance of the infection and confers immunity by killing, which is a form of immunotherapy of any microorganisms that eventually infect the vaccinated person.

The invention thus allows the use of a vaccine to prevent and to treat an established infection by any microorganism which has had its lipid coat removed in vitro to expose its true antigen.

Viruses are believed to have arisen from the nucleic material of pre-existing cells. Although they exist as separate entities, viruses depend entirely on living cells for their vital functions and survival. They have not therefore been exposed to the hostile effects of the sun as were free-living microorganisms. Those viruses that have evolved into chronic infections in man and animals such as the HIV and other retroviruses, the Hepatitis B virus, the Hepatitis C virus, the EBV, the CMW, the Herpes simplex virus, and the tumor-causing viruses in man and animals are all enveloped viruses. The viral envelopes have been acquired from their host cells in the course of their evolution to ensure their survival in the body.

The DNA viruses such as the EBV, CMV and Hepatitis B viruses acquired their envelopes from the nuclear membrane of the host cell as they leave the nucleus from which they developed. The RNA viruses such as the retroviruses and Hepatitis C virus acquired their respective envelopes from the cell wall membrane as the mature virus finally leaves the host cell in which it has developed. Because they carry envelopes that are of host cell origin, enveloped viruses are perceived as ‘hybrid’ viruses, combining viral cores that are foreign to the body with envelopes that are of host origin. For that reason, enveloped viruses are perceived by the host immune system as ‘partly self.’

An effective immune response by the host that killed the virus with its envelope would also kill all those host cells from which the viral envelope was derived. In the case of Hepatitis B and C, the liver cells would be killed as well. In the case of HIV, all the CD4+ cells would be eliminated. In all such situations, this would lead to serious and fatal autoimmune diseases. To avoid killing the body along with the virus the immune response of the host is therefore obliged to be ineffective. In taking its envelope from the host cell membranes, these viruses have ‘deliberately’ taken important ‘hostages’ with which to ‘blackmail’ the immune systems of the body into ineffective responses. These ineffective immune responses ensure the persistence of the enveloped viruses as chronic infections in the body.

The viral envelope, like the cell membrane from which it is derived, contains phospho-lipoproteins that can be easily destroyed with lipid solvents such as chloroform or ether. When the envelope has been destroyed, the rest of the viral antigens, now considered a vaccine when injected into an uninfected person, will be perceived by the immune system as non-self and completely foreign. Appropriate immune responses are provoked that are effective in destroying only the viral cores or non-enveloped parts of the virus, in keeping with the viral antigens used in the vaccine. Any subsequent infection of the vaccinated body by the same enveloped virus will have only its core destroyed leaving its envelope intact. Empty envelopes have no biological importance in the person. The vaccinated person would be immune against the enveloped virus concerned. By destroying the envelopes of enveloped viruses, the rest of the viral antigens constitute a valid and effective vaccine for the virus concerned. Administered to an uninfected person, such a vaccine will achieve a permanent preventive immunity by destroying only the viral core of any subsequent enveloped virus of the same type that infects the body.

In an established infection by an enveloped virus, the host nature of the envelope on the virus blackmails the natural killer cells and blocks any effective long-term immune responses that could kill the virus. However, when the viral envelope is destroyed in vitro and the rest of the antigens re-injected as a vaccine into persons with the established infection, they are perceived as completely foreign and non-self. The vaccine provokes immediate cytokines that will penetrate the viral envelopes and cell membranes containing enveloped viruses and kill the viruses within. The amount of the cytokines produced will depend on the amount of the vaccine used and the state and number of the natural killer cells in the patients. When the infection has been eliminated or reduced to insignificant levels, a long-lasting preventative vaccination or immunotherapy of the patient is achieved as follows.

After verifying that the patient's immune system is competent and that viruses have been eliminated or reduced significantly (in the case of HIV, a count of CD4+ above 400-500, and a viral count below 50 copies/ml), heparine blood is withdrawn from the patient. The peripheral leucocytes in the blood are washed free from all traces of antibodies to the virus. The immune competent cells in the washed leucocytes not committed to the virus are suspended with others in a medium containing the patient's serum from which immunoglobulins and antibodies have been removed. (Cord serum with no antibodies to the virus can replace the patient's antibody-free serum. The serum in the mixture ensures that the antigens of the vaccine are properly presented to the immune system.) The vaccine is added to this mixture and after incubation is it re-injected into the patient. This is immunotherapy of the patient and is similar to the injection of a preventive vaccine into an uninfected person with a normal immune system having no infection or antibodies to the virus.

The re-injected mixture will continue in vivo the process that was started in vitro and the new immune responses provoked by the immunotherapy eliminates from the body any remnants of viruses and prevent any future re-infection by the same enveloped virus. Thus a vaccine can be prepared by destroying the envelope of any given enveloped virus and can be used to prevent or treat an infection by the same enveloped virus.

All immune responses in the body utilize specific complement in one form or another. Even where the immune response is ineffective, as in most chronic infections by microorganisms and enveloped viruses, complement is used all the same. In time, complement specific to the microorganism or virus is in short supply having been used up in a futile attempt to eliminate the chronic infection.

The immunotherapy started in vitro that provokes new and effective immune responses in the patient will also require complement. This new demand for complement can only aggravate the existing chronic shortage and worsen the clinical situation of the patient. It is therefore essential that the viral load or the microorganism be substantially reduced in the patient by other means so that the complement produced thereafter is available for completing the new and effective immune responses provoked by the immunotherapy started in vitro as described above.

One method for reducing the viral load of microorganisms, as mentioned above, is to start by first giving moderately large doses as simple direct injections into the patient of the vaccine with true antigens exposed with viral envelopes removed. This occurs where the new system is still competent. These do not really vaccinate the patient but provoke the natural killer cells to produce cytokines that should kill large numbers of the viruses or microorganisms. The vaccine is then similar to a drug. If repeated at suitable intervals, this killing can significantly reduce the viral load or microorganisms and their need for complement to insignificant levels. The complement produced or freed in the body after that is then available for the subsequent immunotherapy started in vitro that provokes effective immune responses in the body. This latter step is carried out only when there is evidence that the viral load of microorganisms in the patient do not make significant demands on complement.

Where the immune system is depressed, a culture of the washed leucocytes of the patient with the vaccine given to the patient with the vaccine for a period of time will provide the cytokines which when re injected into the patient will provoke a fall in the viral count or microorganisms. This can be repeated 2 or 3 times within a month.

Another quick method for ensuring that the immunotherapy started in vitro is effective in the patient with an ongoing chronic infection is to provide extra complement by the transfusion of fresh blood to the patient. The transfusion is most useful when the new immune responses begin to appear, usually after about 10-14 days following the immunotherapy. The blood transfusion should be repeated until the virus or microorganism is completely eliminated.

### DETAILED DESCRIPTION OF THE INVENTION

Thus, according to a first aspect of the invention, there is provided a method of making a vaccine, the method including the steps of:

extracting a biological fluid obtained from a person or animal infected with a lipid-containing infectious organism with a lipid-containing solvent, the biological fluid containing the lipid-containing infectious organism and the extraction producing an aqueous phase and a lipid-containing organic phase, the aqueous phase containing the infectious organism with the lipid substantially removed, and

isolating the aqueous phase.

The lipid-extracting solvent may be chloroform or ether.

The biological fluid may be whole blood, plasma, pleural fluid, cerebrospinal fluid, culture fluid or other localized body fluid. The lipid-containing infectious organism may be a virus, a bacterium, for example a tuberculosis or leprosy bacterium, a protozoa, a fungus or a mold of the type which contains lipid or lipid-like material in its cell wall. In particular, it may be an enveloped virus such as an immunodeficiency virus for example the human immunodeficiency virus, or HIV. It may, instead, be an enveloped virus such as Hepatitis B, Hepatitis C, Epstein-Barr, Herpes simplex, a cytomegalovirus, a human T-cell leukemia virus (HTLV1), a cancer virus or any other similar enveloped virus.

The extraction process of the biological fluid containing the lipid-containing infectious organism may be by mixing, swirling, vortexing, rotating or by any other suitable process which would be familiar to the person skilled in the art. The extraction time will be sufficient to allow the chloroform to solubilize all, or substantially all, of the lipid contained in the infectious organism and will vary with the extraction process. For example in the case of vortex mixing, such as in the case of HIV, the vortexing may be carried out over a period of about 30-60 minutes and typically about 30 minutes.

The method may include the further step of concentrating the aqueous phase to produce a concentrate containing the infectious organism or of otherwise isolating the infectious organism with the lipid substantially removed, for example by lyophilisation or by any other technique which would be familiar to the person skilled in the art.

The vaccine produced in this way may be used as an auto-vaccine for the person from whom the biological fluid was extracted. Instead, the method may include the additional step of isolating and culturing the infectious organism from an infected person inhabiting a particular geographical region. In this form, the vaccine may be used either prophylatically (as a true vaccine) or for the treatment of persons from that geographical region. This aspect of the invention is particularly important in the case of a virus such as HIV which differs in form in different geographical areas.

The method may include the step of diluting the biological fluid or of diluting the aqueous extract in order to obtain antigens from approximately 100-200 viral particles per ml. Of vaccine

The ratio (volume:volume) of the chloroform to the biological fluid used in the extraction step may be from about 3:1 to about 5:1. It is preferably about 3:1.

According to another aspect of the invention, there is provided a method of making a first therapeutic vaccine. This method includes the steps of:

extracting, with a lipid-extracting solvent, a biological fluid obtained from a person or animal infected with a lipid-containing infectious organism, the biological fluid containing the lipid-containing infectious organism, and the extraction producing an aqueous phase and a lipid-containing phase, the aqueous phase containing the infectious organism with the lipid substantially removed,

separating the aqueous phase from the lipid-containing phase,

isolating a leukocyte fraction from the blood of the person or animal, the isolation being conducted so that the leukocyte fraction contains substantially no plasma, free lipid-containing infectious organism or free antibodies to the lipid containing infectious organism, and

combining at least some of the aqueous phase with at least some of the leukocyte fraction to produce the vaccine.

It will be appreciated that a part of the leukocyte fraction will comprise infected cells.

The first therapeutic vaccine prepared as described above will be used on the patient from whom the biological fluid and the leukocyte fraction were obtained. However, in a development of this embodiment of the invention, the leukocyte fraction from an infected person in a particular geographical area may be combined with a vaccine prepared from isolated cultured virus obtained from another infected person from that same geographical area as described above to produce a second therapeutic vaccine.

Thus the invention extends to a method of making a second therapeutic vaccine. This method includes the steps of:

isolating and culturing an infectious organism from a biological fluid obtained from at least one person or animal infected with a lipid-containing infectious organism to produce a composition containing the cultured lipid-containing infectious organism,

extracting, with a lipid-extracting solvent, an aqueous solution of the lipid-containing infectious organism, the extraction producing an aqueous phase and a lipid containing phase, the aqueous phase containing the organism with the lipid substantially removed,

separating the aqueous phase from the lipid-containing phase,

isolating a leukocyte fraction from the blood of another person or animal infected with the lipid-containing infectious organism, the isolation being conducted so that the leukocyte fraction contains substantially no plasma, free lipid-containing infectious organism or free antibodies to the lipid-containing infectious organism, and

combining at least some of the aqueous phase with at least some of the leukocyte fraction to produce the second therapeutic vaccine.

The lipid-extracting solvent may be any suitable solvent and may be a chlorinated hydrocarbon such as chloroform. It may, instead, be a hydrocarbon or an ether. In particular it is noteworthy that chloroform is suitable for use in the method of the invention even though PCT/IB01/01099 clearly indicates that chloroform denatures many plasma proteins and is unsuitable for use with fluids which will subsequently be administered back to the animal or human.

The leukocyte fraction may be obtained by withdrawing a blood sample from the person or animal, separating the red blood cells from the plasma by sedimentation, separating the leukocytes from the plasma, for example by centrifugation, and washing the leukocytes free of residual plasma and antibodies with normal saline.

The second therapeutic vaccine will be used on the patient from whom the leukocyte fraction was obtained.

In another embodiment of the invention, the aqueous phase produced from the extraction of the cultured lipid-containing infectious organism can be used directly as a prophylactic vaccine for the normal non-infected persons living in the geographical region in which the person or animal from which the biological fluid was obtained and in which the lipid-containing infectious organism is prevalent.

According to another aspect of the invention, there is provided a vaccine prepared by a method which includes the steps of extracting a biological fluid obtained from a person or animal infected with a lipid-containing infectious organism with chloroform, the biological fluid containing the lipid-containing infectious organism, and the extraction producing an aqueous phase and a lipid-containing phase, the aqueous phase containing the infectious organism with the lipid substantially removed, and

isolating the aqueous phase.

According to another aspect of the invention, there is provided a first therapeutic vaccine prepared by a method which includes the steps of:

extracting, with a lipid-extracting solvent, a biological fluid obtained from a person or animal infected with a lipid-containing infectious organism, the biological fluid containing the lipid-containing infectious organism, and the extraction producing an aqueous phase and a lipid-containing phase, the aqueous phase containing the infectious organism with the lipid substantially removed,

separating the aqueous phase from the lipid-containing phase,

isolating a leukocyte fraction from the blood of the person or animal, the isolation being conducted so that the leukocyte fraction is substantially free of plasma, free lipid-containing infectious organism and free antibodies to the lipid-containing infectious organism, and

combining at least some of the aqueous phase with at least some of the leukocyte fraction to produce the vaccine.

According to another aspect of the invention, there is provided a second therapeutic vaccine prepared by a method which includes the steps of:

isolating and culturing an infectious organism from a biological fluid obtained from a first person or a first animal infected with a lipid-containing infectious organism to produce a composition containing the cultured lipid-containing infectious organism,

extracting, with a lipid-extracting solvent, an aqueous solution of the lipid-containing infectious organism, the extraction producing an aqueous phase and a lipid-containing phase, the aqueous phase containing the organism with the lipid substantially removed,

separating the aqueous phase from the lipid-containing phase,

isolating a leukocyte fraction from the blood of a second person or a second animal infected with the lipid-containing infectious organism, the isolation being conducted so that the leukocyte fraction is substantially without plasma, free lipid-containing infectious organism or free antibodies to the lipid-containing infectious organism, and

combining at least some of the aqueous phase with at least some of the leukocyte fraction to produce the second therapeutic vaccine.

According to another aspect of the invention, there is provided a method of treating a person or animal infected with a lipid-containing infectious organism, the method including the steps of:

extracting a biological fluid obtained from a person or an animal infected with a lipid-containing infectious organism with chloroform, the biological fluid containing the lipid-containing infectious organism, and the extraction producing an aqueous phase which contains the infectious organism with the lipid substantially removed and a lipid-containing organic phase,

isolating the aqueous phase, and

administering an effective amount of the aqueous phase to the person or animal.

The method may include the further step of concentrating the aqueous phase to produce a concentrate or of otherwise isolating the infectious organism with the lipid substantially removed as hereinbefore described and administering the resulting substance, optionally with a pharmaceutically acceptable adjuvant or carrier, to the person or animal.

According to another aspect of the invention, there is provided a method of treating a person or an animal infected with a lipid-containing infectious organism, the method including the steps of:

extracting, with a lipid-extracting solvent, a biological fluid obtained from the person or animal, the biological fluid containing the lipid-containing infectious organism, and the extraction producing an aqueous phase which contains the infectious organism with the lipid substantially removed and a lipid-containing phase,

separating the aqueous phase from the lipid-containing phase,

isolating a leukocyte fraction from the blood of the person or animal, the isolation being conducted so that the leukocyte fraction is substantially without plasma, free lipid-containing infectious organism and free antibodies to the lipid-containing infectious organism,

combining at least some of the aqueous phase with at least some of the leukocyte fraction to produce a composition, and

administering a therapeutically effective amount of the composition to the person or animal.

According to another aspect of the invention, there is provided a method of treating a person or animal infected with a lipid-containing infectious organism, the method including the steps of:

isolating and culturing an infectious organism from a biological fluid obtained from a first person or a first animal infected with a lipid-containing infectious organism to produce a composition containing the cultured lipid-containing infectious organism,

extracting, with a lipid-extracting solvent, an aqueous solution of the lipid-containing infectious organism, the extraction producing an aqueous phase which contains the infectious organism with the lipid substantially removed and a lipid-containing phase,

separating the aqueous phase from the lipid-containing phase,

isolating a leukocyte fraction from the blood of a second person or a second animal infected with the lipid-containing infectious organism, the isolation being conducted so that the leukocyte fraction is substantially without plasma, free lipid-containing infectious organism or free antibodies to the lipid-containing infectious organism,

combining at least some of the aqueous phase with at least some of the leukocyte fraction to produce a composition, and

administering a therapeutically effective amount of the composition to the person or animal.

The preceding method may include the step of subjecting the person or animal to a fresh blood transfusion to increase the complement or complement-like proteins in the blood. The complement system is composed of a group of serum proteins that play a major role in the defensive immune response (Microbiology, 2^{nd} Ed, Prescott, Harley and Klein) and the blood transfusion increases the levels of the proteins in a person or animal with a severely compromised immune system.

According to another aspect of the invention, there is provided a third therapeutic vaccine which includes leukocytes which have been exposed in vitro to a vaccine produced by the substantial removal of the lipid portion of a lipid-containing infectious organism, the exposure taking place substantially in the absence of the free lipid-containing infectious organism and in the presence of a medium containing serum which is also free of antibodies to the lipid containing infectious organism.

The leukocytes will typically be exposed at about 37° C. for about 4 hours.

The serum proteins provide opsonins which enable the therapeutic vaccine to achieve performance in the body.

According to another aspect of the invention there is provided a prophylactic vaccine prepared by a method which includes the steps of:

culturing a biological fluid obtained from a person or animal infected with a lipid-containing infectious organism,

extracting the culture containing the lipid-containing organism with a lipid-extracting solvent, the extraction producing an aqueous phase which contains the infectious organism with the lipid substantially removed and a lipid-containing phase, and

isolating the aqueous phase.

The invention extends to a substance or composition for use in a method of treatment of an infection caused by a lipid-containing infectious organism the substance or composition comprising a vaccine or a first or a second or third therapeutic vaccine as hereinbefore described.

The invention extends further to a substance or composition for use in vaccinating a person or animal against an infection caused by a lipid-containing infectious organism, the substance or composition comprising an isolated and cultured infectious organism as hereinbefore described.

The infectious organism may include, HIV, Aghbs, Acv, EBV, Herpes Simplex and tumor viruses with its lipid envelope substantially removed.

The invention extends further to a substance or composition for use in the prophylaxis of an infection caused by a lipid-containing infectious organism the substance or composition comprising a prophylactic vaccine as hereinbefore described.

The invention extends further to the use of a substance or a composition in the manufacture of a medicament for use in the treatment of an infection caused by a lipid-containing infectious organism, the substance or composition comprising a vaccine or a therapeutic vaccine as hereinbefore described.

The invention extends further to the use of a substance or composition in the manufacture of a medicament for use as a vaccine for the prophylaxis of an infection caused by a lipid-containing infectious organism, the substance or composition comprising an isolated and cultured infectious organism as hereinbefore described.

The invention extends further to the use of a substance or composition in the manufacture of a medicament for use in the prophylaxis of an infection caused by a lipid-containing infectious organism, the substance or composition comprising a prophylactic vaccine as hereinbefore described.

The invention is now described, by way of example, with reference to the following Examples, the Table and the Annexes.

### EXAMPLE 1

### The Vaccine

Fresh Fasting blood (5 ml) from an HIV sero-positive person (containing 10,000 to 20,000 viral particles per ml) was withdrawn into a syringe with 500 units of heparine and allowed to stand at room temperature for 30 minutes. This plasma (1 ml) was combined with chloroform (5 ml) in a sterile screw-capped glass tube. The mixture was mixed using a vortex mixer for 5 minutes and then allowed to stand at room temperature for another 25 minutes. Normal saline (9 ml) was added and the mixture was mixed using the vortex mixer for 5 minutes and allowed to stand at room temperature for 10 minutes. The resulting mixture was transferred to a sterile screw-capped glass centrifuge tube and centrifuged for 15 minutes at 4000 rpm. The aqueous supernatant was transferred to a sterile glass Petri dish with a base diameter of 10 cm and allowed to stand under an extraction hood for 30 minutes. The resulting 10 ml of vaccine constitutes 10 doses and contained antigens from 1,000-2,000 viral particles per ml of vaccine. The first vaccine was stored at minus −200° C. as a single dose.

### EXAMPLE 2

### The Vaccine

In another embodiment of the invention, fresh fasting blood (5 ml) from a patient with 100,000-200,000 viral particles per ml was drawn into a syringe with 500 units of heparine. This plasma (1 ml) was combined with chloroform (5 ml) in a sterile screw-capped glass tube and thoroughly mixed with a vortex mixer for 10-15 minutes. The mixture was allowed to stand at room temperature (20-25° C.) for 45 minutes. Normal saline (9 ml) was added to the mixture and vortexed for 5 minutes and allowed to stand for 10 minutes at room temperature. The mixture was transferred into sterile screw-capped centrifuged tubes and centrifuged at 4000 rpm for 15 minutes. The supernatant was transferred to a sterile glass Petri dish with a base diameter of 10 cm and allowed to stand under an extraction hood for 30 minutes. The supernatant (1 ml) was diluted with normal saline, (9 ml) to give a dilution of 1/100. This final dilution (1 ml) contained antigens from about 1,000-2,000 viral particles per ml and was stored in single doses at minus −20° C.

### EXAMPLE 3

### The Effect of Auto-Vaccine on Viral Counts (See Table 1, Annex 1)

Auto-vaccines were prepared according to the method of Example 1, and contained antigens from 1/100 the viral count in the patient are concerned. The auto-vaccine (1 ml) was subcutaneously injected directly into the patient. This produced significant falls of viral counts in the patient concerned except in patient no. 7. In patient nos. 1 and 2, the auto-vaccine was injected on 2 separate occasions 4 days apart and the absolute fall in viral count was 91% and 75% respectively.

In another patient (Annex 1 (b)), 1 ml of auto-vaccine was administered as 5 simple s/c injections on 17 Apr. 2001, 21 Apr. 2001, 21 Jun. 2001, 21 Aug. 2001 and 21 Oct. 2001. The viral count fell from 29,566 (4.5 log₁₀) on 16 Apr. 2001 to below 50 (1.5log₁₀) on 2 Feb. 2002 15 months later and the HIV serology was indeterminate. However on 17 Dec. 2002 the viral count rose to 65 (1.8log₁₀). This suggests that the auto-vaccine administered as a direct injection caused the viral count to fall to the point where the HIV serology was indeterminate but without further auto-vaccines, the viral replication restarted.

### EXAMPLE 4

### The Effect of Auto-Vaccine with Washed Peripheral Leukocytes (See Annex 2)

The auto-vaccine was prepared according to the method of Example 1 and diluted 1/100. A blood sample (20 ml) was withdrawn into a syringe with 1,000 units of heparine. The sample was transferred to a long sterile screw-capped tube with a base diameter of 1 cm and allowed to stand upright for 30 minutes at −37° C. The leukocyte-rich plasma above the red cell sediment was aspirated off and centrifuged at 2,000 rpm for 10 minutes in a bench centrifuge (Jouan C312). The supernatant plasma was discarded and normal saline (5 ml) was added to the leukocyte deposit and after vortexing for 1 minute the mixture, again centrifuged at 2,000 rpm for 10 minutes.

The supernatant was discarded and fresh normal saline (5 ml) was added to the deposit. This process of washing the leukocytes was repeated 2 more times to remove all traces of antibodies from the cell deposit. The auto-vaccine (1 ml) was then added to the washed leukocytes and the mixture injected immediately subcutaneously into the patient concerned. The procedure was repeated on the dates shown in Table 2 below. There was a marked increase of CD4 in patients and significant falls of viral counts in all patients and was below 50 particles in 9 of 34 patients.

### EXAMPLE 5

### The Therapeutic Vaccine and Immunotherapy

The washed peripheral leukocytes were prepared as in Example 4. The auto-vaccine (1 ml), prepared according to the method of Example 1, was added to the cell deposit followed by culture medium of RPMI (GIBCO) (5 ml) with 10% human cord serum free from antibodies to HIV and other viruses in place of the patient's serum from which immunoglobins have been removed. The mixture was incubated at 37° C. for 4 hours. The therapeutic vaccine was re-injected directly subcutaneously into the forearm of the person from whom the blood sample has been withdrawn and auto-vaccine prepared. The whole procedure was repeated on day 5 and day 30 as a booster.

A mild immune reaction in the form of mild tiredness, body aches and a mild fever lasting a few day occurred in a few patients about 10-14 days from the first immunotherapy. These symptoms were controlled by anti-pyretics and anti-inflammatory drugs such as aspirin, and dexamethazone.

When these symptoms persist, they were usually stopped by a transfusion of fresh blood which provided additional complement for the immune reaction provoked by the therapeutic vaccine and immunotherapy. The CD4+ and viral counts were repeated every 3-4 months until the viral counts were below detection levels. Monitoring was continued annually for three years. In a few cases, the sero-positive status of the patient converted to HIV sero-negative after varying periods.

The results of the treatment in Example 5 on five patients are set out in Table 1 below.

| Patient No. | 1 | 2 | 3 | 4 | 5 |
|---|---|---|---|---|---|
| CD4 before | 342 | 210 | 214 | 350 | 846 |
| treatment | | | | | |
| (cells/ml) | | | | | |
| CD4 after | 424 | 443 | 587 | 1537 | 910 |
| treatment | | | | | |
| cells/ml | | | | | |
| Viral load | 23200 | 31460 | 42300 | 17300 | 207,743 |
| before | | | | | |
| treatment | | | | | |
| Viral load after | 505 | 4005 | 3218 | Less than 50 | Less than 50 |
| treatment | | | | | |
| Serology | + | + | + | −ve | −ve |
| Time (months) | 5 | 4 | 7 | 3 | 4 |

The time refers to the number of months after the treatment before the CD4 and viral load were measured again. Viral particles could not be counted below 50.

A sixth patient seen and treated in October 1992 according to Example 5, and for whom CD4+ and viral loads were not measured, reported sero-negative three years later. The sero-negative results in case nos. 4 and 5 above and in the 6^{th} patient confirm that the HIV had been completely eliminated from the patients.

It is believed that the reason why HIV is not eliminated from the body by the immune system is because it is an enveloped virus with the envelope taken from the CD4+ membranes as the mature virus leaves the infected cell. A full discussion of this hypothesis was published by the Applicant in Medical Hypotheses (1994) 42, 81-88 and Medical Hypotheses (1997) 48, 517-521, incorporated by reference herein. Because the envelope of HIV is derived from the CD4+ membrane, the immune system perceives the virus to be “partly self.” An effective immune response, which ignores the presence of the envelope and kills the virus with the envelope, would also kill all the CD4+ cells from which the viral envelope is derived. The HIV has effectively taken a “hostage” in the form of the lipid containing envelope with which it “blackmails” the host immune system into an ineffective response.

An effective preventative vaccine is a prophylactic immunotherapy and must meet three requirements. These are, firstly, that the vaccine must provoke an immune response that kills the virus, secondly, that the immune system of the person must be competent and thirdly that there should be no pre-existing antibodies to the virus in the person to be vaccinated. To be affective, a therapeutic vaccine used as immunotherapy to treat HIV infection after it has occurred must also produce a prophylactic immunotherapy in the HIV infected person. In other words, the HIV vaccine should provoke an immune response that kills the virus in the infected person, the person should have immune competence and antibodies to HIV should be absent from that part of the immune system of the infected person which is to be vaccinated. At the beginning of HIV infection, and for a considerable time thereafter, the person has a competent immune system with several immune units in it. These deal successfully with different infections or antigens that invade the body at any given time. Evidence of this is that infected agents which invade the body in the first few years after HIV infection are successfully controlled by the immune system.

If in vitro one of the immune units not committed to the HIV present in the peripheral blood leukocytes were washed free of HIV antibodies and infectious agent and combined with an effective HIV vaccine, the immune unit would perceive the vaccine as a “non-self” and foreign antigen because of the absence of the viral envelope. An effective immune response appropriate to a “non-self” foreign antigen would start in vitro. When the mixture is re-injected into the infected person, the vaccinated immune cells and their descendants should continue the process in vivo and eventually kill only the core of the HIV in the person. This should eventually eliminate the virus from the body provided there is complement available, as would have happened in a vaccination of a normal uninfected person. An essential feature of the invention is that a part of a person's immune system contained in his peripheral leukocytes can be vaccinated in vitro in a medium free of HIV antibodies and lipid-containing infectious agent before being re-injected into the person.

### ANNEX 1

CHANGE IN VIRAL COUNT 3-4 WEEKS AFTER ONE DOSE OF AUTO-VACCINE

| | | | | | | | | Column 5 | | |
|---|---|---|---|---|---|---|---|---|---|---|
| | | | | Column 3 | | Column 4 | | ABSOLUTE FALL IN | | Column 6 |
| | | Column 2 | | VIRAL COUNT BEFORE | | VIRAL COUNT AFTER | | VIRAL COUNT AFTER | | PERCENTAGE |
| | Column 1 | INITIAL | | AUTO-VACCINE | | AUTO-VACCINE | | THREE-FOUR WEEKS | | FALL IN VIRAL |
| | INITIAL | T4 | T8 | Copies/ml | | Copies/ml | | Copies/ml | | LOAD |
| 1 | NA | 567 | 1302 | 140 362 | (5.1 log₁₀) | 12 152 | (4.1 log₁₀) | 128 110 | (5.1 log₁₀)* | 91.00% |
| 2 | FE | 408 | 955 | 166 476 | (5.2 log₁₀) | 41 157 | (4.6 log₁₀)^{⊕} | 125 319 | (5.1 log₁₀)* | 75.00% |
| 3 | CH | 433 | >2000 | 220 860 | (5.0 log₁₀) | 147 073 | (5.2 log₁₀) | 80 787 | (4.9 log₁₀) | 36.00% |
| 4 | FM | 505 | 339 | 46 416 | (4.7 log₁₀) | 28 194 | (4.5 log₁₀) | 18 222 | (4.3 log₁₀) | 39.30% |
| 5 | SM | 390 | 1370 | 13 358 | (4.1 log₁₀) | 4 047 | (3.6 log₁₀) | 9 301 | (4.0 log₁₀) | 69.60% |
| 6 | BD | 667 | 969 | 6 524 | (3.8 log₁₀) | 1 098 | (3.0 log₁₀) | 5 426 | (3.7 log₁₀) | 86.20% |
| 7 | CD | 387 | 424 | 69 192 | (4,8 log₁₀) | 65 029 | (4.8 log₁₀) | 4 163 | (3.6 log₁₀) | 6.00% |
| 8 | NNG | 364 | 845 | 13 056 | (4.1 log₁₀) | 4 517 | (3.6 log₁₀) | 8 739 | (3.9 log₁₀) | 66.93% |
| 9 | LG | 739 | 847 | 3 073 | (3.5 log₁₀) | 1 800 | (3.3 log₁₀) | 1 273 | (3.1 log₁₀) | 41.40% |
| 10 | AD | 554 | 1110 | 2 714 | (3.4 log₁₀) | 1 563 | (3.2 log₁₀) | 1 151 | (3.1 log₁₀) | 42.00% |

*Received 2 doses of vaccines.
^{⊕}Viral count 4 weeks after auto-vaccine.

### ANNEX 1B

D. B. M/Male 51 Years

| Apr. 19, 2001 | Serology HIV |
|---|---|
| Apr. 14, 2001 | VC 29,566 (4.5 log₁₀) |
| Apr. 17, 2001 | T3 1181 |
| | T4 877 |
| | T8 344 |
| | T4/T8 2.2 (CPC N^{o} 13,502) |
| 1 ml plasma/leucocytes @ 40° C. 24 hrs chloroform | |
| 1 ½ hrs + 9 ml ml N/S 15 mins | |
| centrifuged. Supernatant 1/100 = Vaccine | |
| Apr. 17, 2001 | 1 ml vaccine s/c. |
| Apr. 21, 2001 | 1 ml vaccine s/c. |
| Jun. 21,2001 | 4 doses of vaccine given every 2 months. |
| Aug. 21, 2001 | |
| Oct. 21, 2001. | |
| Dec. 13, 2001 | T3 1496 |
| | T4 1007 |
| | T8 453 |
| | T4/T8 2.4 |
| | VC <50 (1.7 log₁₀) |
| | Serology indeterminate (CPC 22,443) |
| Dec. 17, 2002 | T3 1664 |
| | T4 1091 |
| | T8 539 |
| | T4/T8 2.0 |
| | VC 65 (1.8 log ₁₀) |

### ANNEX 2

AUTO-VACCINATION WITH WASHED PERIPHERAL LEUKOCYTES

| | | | | | Viral load | Viral load | |
|---|---|---|---|---|---|---|---|
| | | | CD4 before | CD4 after | before vaccine/ | After vaccine/ | |
| N^{o} | Folder No | Age/Sex | Vaccine/Date | Vaccine/Date | Date | Date | % fall in Viral load |
| 1 | 474 | 34 yrs/F | 384 | 357 | 2314 | <50 | 97% |
| | | | (Jun. 10, 2003) | (Dec. 19, 2003) | (Jun. 7, 2003) | (Dec. 4, 2003) | |
| 2 | 456 | 23 yrs/F | 1021 | 567 | 786 | <50 | 97% |
| | | | (Jan. 16, 2003) | (Apr. 17, 2003) | (Jan. 16, 2003) | (Dec. 4, 2003) | |
| 3 | 666 | 4 yrs/F | 713 | 625 | 546 | <50 | 97% |
| | | | (Feb. 01, 2003) | (Aug. 21, 2003) | (Feb. 11, 2003) | (Oct. 30, 2003) | |
| | | | 694 | 694 | 20641 | | |
| | | | | (Oct. 30, 2003) | (Aug. 20, 2003) | | |
| 4 | 1102 | 26/ | 225 | | 16877 | <50 | 97% |
| | | | (Mar. 10, 2003) | | (Jun. 17, 3003) | (Dec. 4, 2003) | |
| 5 | 633 | 32 yrs | 785 | | 2314 | <50 | 97% |
| | | | (Aug. 7, 2003) | | (Aug. 7, 2003) | (Dec. 4, 2003) | |
| 6 | 471 | 30 yrs/F | 771 | 963 | 3778 | <50 | 97% |
| | | | (May 27, 2003) | (Oct. 28, 2003) | (May 27, 2003) | Oct. 28, 2003 | |
| 7 | B. M | 25 yrs/F | 200 | 233 | 272,902 | <50 | 97% |
| | | | (Jul. 12, 2003) | (Aug. 13, 2002) | (Sep. 6, 2001) | (Aug. 13, 2002) | |
| 8 | 1667 | 29 yrs/F | 761 | 790 | 71 | <50 | 97% |
| | | | (Jun. 03, 2003) | (Oct. 7, 2003) | (Jun. 3, 2003) | (Sep. 10, 2003) | |
| 9 | 1521 | 35 yrs/F | 839 | 912 | 129 | <50 | 97% |
| | | | (Nov. 14, 2002) | (Sep. 2, 2003) | (Nov. 14, 2002) | (Sep. 2, 2003) | |
| 10 | 417 | 39 yrs/F | 279 | 300 | 172652 | 13668 | 97% |
| | | | (Jul. 23, 2003) | (Nov. 6, 2003) | (Apr. 1, 2003) | (Nov. 27, 2003) | |
| 11 | 822 | 47 yrs/F | 238 | | 30430 | 371 | 98% |
| | | | (Nov. 8, 2003) | | (Dec. 12, 2002) | (Dec. 5, 2003) | |
| 12 | 400 | 20 yrs/F | 424 | 499 | 30486 | 6197 | 79% |
| | | | (May 27, 2003) | (Nov. 27, 2003) | (May 30, 2003) | (Nov. 21, 2003) | |
| 13 | 1584 | | 939 | 836 | 15402 | 341 | 97% |
| | | | (Jul. 10, 2003) | (Nov. 18, 2003) | (Jul. 10, 2003) | (Nov. 18, 2003) | |
| 14 | 2106 | 27 yrs | 687 | 758 | 59602 | 28646 | 51% |
| | | | (Jul. 24, 2003) | (Nov. 27, 2003) | (Sep. 9, 2003) | (Nov. 27, 2003) | |
| 15 | 1036 | 30 yrs/M | 867 | 496 | 172652 | 13668 | 92% |
| | | | (Mar. 20, 2003) | (Nov. 6, 2003) | (Apr. 1, 2003) | Nov. 6, 2003 | |
| 16 | 387 | 38 yrs/F | 941 | 1116 | 4882 | 1380 | 71% |
| | | | (Jul. 15, 2003) | (Oct. 9, 2003) | (Jul. 2, 2003) | (Oct. 9, 2003) | |
| 17 | 989 | 20 yrs/ | 742 | 852 | 30086 | 3886 | 87% |
| | | | (Oct. 22, 2002) | (Nov. 27, 2003) | (Oct. 22, 2002) | (Oct. 23, 2003) | |
| 18 | 1577 | 40 yrs/F | 362 | 456 | 6355 | 1369 | 78% |
| | | | (Jun. 11, 2003) | (Sep. 25, 2003) | (Apr. 1, 2003) | (Sep. 25, 2003) | |
| 19 | 1049 | 33 yrs/F | 267 | 435 | 125,358 | 10,283 | 92% |
| | | | (Mar. 20, 2003) | (Oct. 28, 2003) | (Mar. 27, 2003) | (Oct. 28, 2003) | |
| 20 | 1936 | 38 yrs/M | 1016 | 980 | 2,878 | 157 | 94% |
| | | | Jul. 17, 2003 | Nov. 27, 2003 | Aug. 17, 2003 | Nov. 24, 2003 | |
| 21 | 0070/0670 | 36 yrs/F | 693 | 712 | 1731 | 116 | 93% |
| | | | Mar. 25, 2003 | (Jul. 22, 2003) | Mar. 15, 2003 | Dec. 4, 2003 | |
| 22 | 0803 | — | 241 | 349 | 41,930 | 2,890 | 93% |
| | | | Dec. 10, 2002 | Nov. 27, 2003 | Dec. 10, 2002 | Nov. 27, 2003 | |
| 23 | 0720 | 27 yrs/F | 594 | 607 | 94,228 | 9,541 | 89% |
| | | | Jan. 9, 2003 | Dec. 2, 2003 | Jan. 9, 2003 | Dec. 2, 2003 | |
| 24 | 1747 | 29 yrs/M | 263 | 271 | 32,941 | 6,332 | 80% |
| | | | Feb. 27, 2003 | Nov. 18, 2003 | Feb. 27, 2003 | Nov. 18, 2003 | |
| 25 | 2005 | 19 yrs/F | 455 | 492 | 4,143 | 1,032 | 88% |
| | | | Mar. 7, 2002 | (Jul. 5, 2002) | (Mar. 7, 2002) | (Oct. 17, 2002) | |
| | | | | 579 | | 480 | |
| | | | | (Oct. 14, 2002) | | (Sep. 2, 2003) | |
| | | | | 613 | | | |
| | | | | (Sep. 18, 2003) | | | |
| 26 | 1010 | 35 yrs/F | 898 | 1024 | 35,801 | 92,041 | 91% |
| | | | Feb. 20, 2003 | Oct. 13, 2003 | Feb. 20, 2003 | Oct. 13, 2003 | |
| | | | | 650 | | 2,888 | |
| | | | | Jan. 22, 2004 | | Jan. 22, 2004 | |
| 27 | 851 | 28 yrs/F | 381 | 446 | 55,665 | 11,133 | 94% |
| | | | Feb. 18, 2003 | Oct. 28, 2003 | Feb. 18, 2003 | Nov. 4, 2003 | |
| | | | | 343 | | 3,112 | |
| | | | | Feb. 5, 2004 | | Feb. 5, 2004 | |

What is claimed is:

1. A method of making a vaccine comprising the steps of:
obtaining a biological fluid containing a lipid-containing infectious organism from a person or an animal infected with said lipid-containing infectious organism;
extracting said biological fluid with a lipid-extracting solvent, said extraction producing an aqueous phase and a lipid-containing organic phase, said aqueous phase containing said infectious organism; and
isolating said aqueous phase.

2. The method of claim 1, wherein said biological fluid is selected from the group consisting of whole blood, plasma, pleural fluid, cerebrospinal fluid, culture fluid, and localized body fluid.

3. The method of claim 1, wherein said lipid-containing infectious organism is selected from the group consisting of viruses, bacteria, protozoa, fungi and molds of the type that contain lipid or lipid-like material in their cell walls.

4. The method of claim 3, wherein said infectious organism is an enveloped virus.

5. The method of claim 4, wherein said enveloped virus is selected from the group consisting of human immunodeficiency virus, Hepatitis B virus, Hepatitis C virus, Epstein-Barr virus, Herpes Simplex virus, Cytomegalovirus, human T-cell leukemia virus and cancer virus.

6. The method of claim 3, wherein said infectious organism is a bacterium selected from the group consisting of tuberculosis bacteria and leprosy bacteria.

7. The method as claimed in claim 1, in which the step of extracting is accomplished by any procedure taken from the group consisting of mixing, swirling, vortexing, and rotating.

8. The method of claim 1, including the step of concentrating said aqueous phase to produce a concentrate containing the infectious organism with the lipid substantially removed.

9. The method of claim 1, including the step of concentrating the aqueous phase by lyophilization.

10. The method of claim 1, including the step of diluting said biological fluid and diluting said aqueous extract in order to obtain antigens from approximately 100-200 viral particles per ml.

11. The method of claim 1, wherein said lipid-extracting solvent is chloroform and in which the ratio (volume:volume) of the chloroform to the biological fluid used in the extraction step is from about 3:1 to about 5:1.

12. The method of claim 1, wherein said lipid-extracting solvent is chloroform and in which the ratio (volume:volume) of the chloroform to the biological fluid used in the extraction step is about 3:1.

13. A method of making a therapeutic vaccine including the steps of:
obtaining a biological fluid containing a lipid-containing infectious organism from a person or an animal infected with said lipid-containing infectious organism;
extracting said biological fluid with a lipid-extracting solvent, said extraction producing an aqueous phase and a lipid-containing phase, said aqueous phase containing said infectious organism with the lipid of said lipid-containing infectious organism substantially removed;
separating said aqueous phase from said lipid-containing phase;
isolating a leukocyte fraction from the blood of said person or animal, said isolation being conducted so that said leukocyte fraction is substantially without plasma, free lipid-containing infectious organism, or free antibodies to said lipid-containing infectious organism; and
combining at least some of said aqueous phase with at least some of said leukocyte fraction to produce the therapeutic vaccine.

14. The method of claim 13, wherein said lipid-extracting solvent is selected from the group consisting of chlorinated hydrocarbons, hydrocarbons, and ethers.

15. The method of claim 13, wherein said lipid-extracting solvent is chloroform.

16. The method of claim 13, wherein said leukocyte fraction is obtained by withdrawing a blood sample from said person or said animal separating the blood cells from the plasma, separating the leukocytes from the plasma and washing the leukocytes free of residual plasma and antibodies.

17. A method of making a therapeutic vaccine according to the steps of:
obtaining a biological fluid containing a lipid-containing infectious organism from a first person or a first animal infected with said lipid-containing infectious organism;
isolating and culturing an infectious organism from said biological fluid to produce a composition, containing cultured lipid-containing infectious organism;
extracting an aqueous solution of said lipid-containing infectious organism with a lipid-extracting solvent, the extraction producing an aqueous phase and a lipid-containing phase, said aqueous phase containing the organism with the lipid substantially removed;
separating the aqueous phase from the lipid-containing phase;
isolating a leukocyte fraction from the blood of a second person or a second animal infected with the same lipid-containing infectious organism of said first person or said first animal, the isolation being conducted so that said leukocyte fraction is substantially without plasma, free lipid-containing infectious organism or free antibodies to the lipid-containing infectious organism; and
combining at least some of said aqueous phase with at least some of said leukocyte fraction to produce the therapeutic vaccine.

18. The method of claim 17, wherein said lipid-extracting solvent is selected from the group consisting of chlorinated hydrocarbons, hydrocarbons and ethers.

19. The method of claim 17, wherein said lipid-extracting solvent is chloroform.

20. The method of claim 17, wherein said leukocyte fraction is obtained by withdrawing a blood sample from said second person or said second animal separating the red blood cells from the plasma, separating the leukocytes from the plasma and washing the leukocytes free of residual plasma and antibodies.

21. A vaccine prepared by the steps of:
obtaining a biological fluid containing a lipid-containing infectious organism from a person or an animal infected with said lipid-containing infectious organism;
extracting said biological fluid, said extraction producing an aqueous phase and a lipid-containing phase, said aqueous phase containing said infectious organism with the lipid substantially removed; and
isolating said aqueous phase.

22. The use of a substance or composition in the manufacture of a medicament for use in the treatment of an infection caused by a lipid-containing infectious organism, the substance or composition comprising a vaccine or a therapeutic vaccine as claimed in claim 21.

23. A therapeutic vaccine prepared by a method including the steps of:
obtaining a biological fluid containing a lipid-containing infectious organism from a person or animal infected with said lipid-containing infectious organism;
extracting said biological fluid with a lipid-extracting solvent, said step of extraction producing an aqueous phase and a lipid-containing phase, said aqueous phase containing said infectious organism with the lipid substantially removed;
separating said aqueous phase from said lipid-containing phase;
isolating a leukocyte fraction from the blood of said person or said animal, said step of isolation being conducted so that said leukocyte fraction is substantially without plasma, free lipid-containing infectious organism, and free antibodies to the lipid-containing infectious organism; and
combining at least some of said aqueous phase with a least some of said leukocyte fraction to produce the vaccine.

24. A therapeutic vaccine prepared by a method including the steps of:
obtaining a biological fluid containing a lipid-containing infectious organism from a first person or a first animal infected with said lipid-containing infectious organism;
isolating and culturing said infectious organism to produce a composition containing the cultured lipid-containing infectious organism;
extracting an aqueous solution of said lipid-containing infectious organism using a lipid-extracting solvent, the extraction producing an aqueous phase and a lipid-containing phase, said aqueous phase including said organism with the lipid substantially removed;
separating said aqueous phase from said lipid-containing phase;
isolating a leukocyte fraction from the blood of a second person or a second animal infected with said lipid-containing infectious organism, said step of isolation being conducted so that said leukocyte fraction is substantially without plasma, free lipid-containing infectious organism or free antibodies to the lipid-containing infectious organism; and
combining at least some of said aqueous phase with at least some of said leukocyte fraction to produce the therapeutic vaccine.

25. A therapeutic vaccine which includes leukocytes which have been exposed in vitro to a vaccine produced by the substantial removal of the lipid portion of a lipid-containing infectious organism, the exposure taking place substantially in the absence of the free lipid-containing infectious organism and in the presence of a medium containing serum which is also free of antibodies to the lipid containing infections organism.

26. A prophylactic vaccine prepared by a method which includes the steps of:
obtaining a biological fluid from a person or an animal infected with a lipid-containing infectious organism;
culturing said biological fluid;
extracting the culture containing the lipid-containing organism using a lipid-extracting solvent, the step of extraction producing an aqueous phase and a lipid phase, said lipid phase containing the infections organism with the lipid substantially removed; and
isolating said aqueous phase.

27. A substance or composition for use in the prophylaxis of an infection caused by a lipid-containing infectious organism, the substance or composition comprising a prophylactic vaccine as claimed in claim 26.

28. The use of a substance or composition in the manufacture of a medicament for use in the prophylaxis of an infection caused by a lipid-containing infectious organism, the substance or composition comprising a prophylactic vaccine as claimed in claim 26.

29. A substance or composition for use in a method of treatment of an infection caused by a lipid-containing infectious organism, the substance or composition comprising a vaccine, or a therapeutic vaccine prepared by a method as claimed in claim 1.
